(19) United States
(12) Patent Application Publication
(10) Pub. No.: US 2026/0248900 A1
(43) Pub. Date: Aug. 27, 2026

(54) MALARIA IMMUNOGEN AND METHODS FOR USING SAME

(71) Applicants: UNM RAINFOREST INNOVATIONS, Albuquerque, NM (US); THE JOHNS HOPKINS UNIVERSITY, Baltimore, MD (US)

(72) Inventors: Bryce C. CHACKERIAN, Albuquerque, NM (US); Fidel P. ZAVALA, Baltimore, MD (US); Alexandra FRANCIAN, Albuquerque, NM (US); Yogesh NEPAL, Albuquerque, NM (US); Bryce T. ROBERTS, Albuquerque, NM (US); Yevel FLORES-GARCIA, Baltimore, MD (US)

(21) Appl. No.: 19/543,984

(22) Filed: Feb. 19, 2026

Related U.S. Application Data
(60) Provisional application No. 63/761,539, filed on Feb. 21, 2025.

(51) Int. Cl. A61K 39/015; A61K 39/00

(52) U.S. Cl. CPC A61K 39/015; A61K 2039/505

(57) ABSTRACT

An immunogen useful for treating malaria generally includes an immunogenic carrier and an antigenic malaria circumsporozoite protein (CSP) peptide that includes the peptide NANPNVDP (SEQ ID NO:2) linked to the immunogenic carrier. The immunogen may be administered to a subject having or at risk of having malaria. Alternatively, the immunogen may be administered to an individual having or at risk of having Plasmodium falciparum blood stage parasitemia. In some cases, the immunogen can be administered in combination with another therapeutic agent for treating malaria of Plasmodium falciparum blood stage parasitemia.

### CROSS REFERENCE TO RELATED APPLICATION

This application claims the benefit of U.S. Provisional Patent Application No. 63/761,539, filed Feb. 21, 2025, which is incorporated herein by reference in its entirety.

### GOVERNMENT FUNDING

This invention was made with government support under AI169739 awarded by the National Institutes of Health. The government has certain rights in the invention.

### SEQUENCE LISTING

This application contains a Sequence Listing electronically submitted via Patent Center to the United States Patent and Trademark Office as an .xml file entitled “0310_000196US01.xml” having a size of 10,395 bytes and created on Feb. 17, 2026. The information contained in the Sequence Listing is incorporated by reference herein.

### SUMMARY

This disclosure describes, in one aspect, an immunogen useful for treating malaria. Generally, the immunogen includes an immunogenic carrier and an antigenic malaria circumsporozoite protein (CSP) peptide that includes NANPNVDP (SEQ ID NO:2) linked to the immunogenic carrier.

In another aspect, this disclosure describes an immunogen useful for treating malaria. Generally, the immunogen includes an immunogenic carrier and an antigenic malaria CSP peptide linked to the immunogenic carrier. The antigenic malaria CSP peptide includes amino acids 1 to 8 of SEQ ID NO:3, amino acids 1 to 9 of SEQ ID NO:3, amino acids 1 to 10 of SEQ ID NO:3, amino acids 1 to 11 of SEQ ID NO:3, amino acids 1 to 12 of SEQ ID NO:3, amino acids 1 to 13 of SEQ ID NO:3, or amino acids 1 to 14 of SEQ ID NO:3. In one or more embodiments, the immunogenic carrier can include a bacteriophage VLP such as, for example, an MS2 bacteriophage VLP or a Qβ bacteriophage VLP.

In one or more embodiments of either aspect, the immunogen further includes a second antigenic malaria CSP peptide. In one or more embodiments, the first antigenic CSP peptide and the second CSP peptide are displayed on a single VLP.

In another aspect, this disclosure describes a composition that includes any embodiment of immunogen described herein.

In one or more embodiments, the composition includes a first population of VLPs displaying SEQ ID NO:2 and a second population of VLPs displaying a second antigenic CSP peptide.

In one or more embodiments, the composition can further include an adjuvant. In one or more of these embodiments, the adjuvant can include an agonist of TLR7, an agonist of TLR8, or an agonist of both TLR7 and TLR8.

In another aspect, this disclosure describes a method of treating malaria in an individual. Generally, the method includes administering to the individual a therapeutically effective amount of a composition that includes any embodiment of immunogen described herein.

In one or more embodiments, the method can further include administering to the individual at least one additional therapeutic agent for treating malaria.

In one or more embodiments, the composition administered to the individual includes a first population of immunogens and a second population of immunogens. The first population of immunogens includes an immunogenic carrier and an antigenic malaria CSP peptide linked to the immunogenic carrier. The antigenic malaria CSP peptide includes the amino acids of SEQ ID NO:2, amino acids 1 to 9 of SEQ ID NO:3, amino acids 1 to 10 of SEQ ID NO:3, amino acids 1 to 11 of SEQ ID NO:3, amino acids 1 to 12 of SEQ ID NO:3, amino acids 1 to 13 of SEQ ID NO:3, or amino acids 1 to 14 of SEQ ID NO:3. The second population of immunogens includes a second population of immunogenic carriers and the second CSP peptide linked to the second population of immunogenic carriers.

In one or more embodiments, the composition is administered to the individual before the individual manifests a symptom or clinical sign of malaria.

In one or more embodiments, the composition is administered to the individual before the individual travels to a geographical location where malaria may be prevalent.

In another aspect, this disclosure describes a polynucleotide encoding any embodiment of immunogen described herein.

In another aspect, this disclosure describes an expression vector that includes any embodiment of polynucleotide described herein.

In another aspect, this disclosure describes a host cell that includes any embodiment of expression vector described herein.

In another aspect, this disclosure describes a vaccine that includes any embodiment of the composition described herein.

In another aspect, this disclosure describes a method of treating malaria in an individual. Generally, the method includes administering to the individual a therapeutically effective amount of any embodiment of vaccine described herein.

In one or more embodiments, the method further includes administering to the individual at least one additional therapeutic agent for treating malaria.

In one or more embodiments, the vaccine is administered to the individual before the individual manifests a symptom or clinical sign of malaria.

In one or more embodiments, the vaccine is administered to the individual before the individual travels to a geographical location where malaria may be prevalent.

In another aspect, this disclosure describes a method of treating Plasmodium falciparum blood stage parasitemia in an individual. Generally, the method includes administering to the individual a therapeutically effective amount of any embodiment of composition described herein.

In one or more embodiments, the method further includes administering to the individual at least one additional therapeutic agent for treating malaria.

In one or more embodiments, the vaccine is administered to the individual before the individual manifests a symptom or clinical sign of Plasmodium falciparum blood stage parasitemia.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Characterization of MS2 L9 VLPs. (A) Structure of the MS2 coat protein dimer (left) and the MS2 VLP (right). 90 coat protein dimers self-assemble into a VLP. The location of the AB-loop is highlighted on both structures. (B) PfCSP domains and amino acid sequence in the junctional repeat region (SEQ ID NO:1) and minor repeat regions, NDPD (SEQ ID NO:8); NVDP (SEQ ID NO:9); and NANP (SEQ ID NO:10). The sequence of the three inserted L9 sequences (SEQ ID ID:2, SEQ ID NO:3, and SEQ ID NO:4) and the location of the core NPNV (SEQ ID NO:11) epitopes (underlined) are shown. (C) SDS-PAGE analysis of wildtype MS2 VLPs and MS2 L9 VLPs displaying the 8 amino acid (SEQ ID NO:2), 15 amino acid (SEQ ID NO:3), and 27 amino acid (SEQ ID NO:4) L9 epitopes. (D) Transmission electron microscopy (TEM) images of recombinant MS2 L9 VLPs. The scale bar (in white) represents 100 nm. (E) Binding of L9 mAb to MS2 L9 VLPs or wildtype MS2 VLPs, as measured by ELISA.

FIG. 2. MS2 L9 VLPs elicit strong anti-CSP antibody responses. (A) Comparison of the immunogenicity of MS2 L9 VLPs displaying eight amino acid (SEQ ID NO:2), 15 amino acid (SEQ ID NO:3), or 27 amino acid (SEQ ID NO:4) L9 epitopes. Groups of Balb/c mice (n=5) were immunized intramuscularly with 5 μg of VLPs at week 0 and week 3. Anti-CSP IgG responses were measured by ELISA using sera collected one week after the second immunization. Results show mean ELISA data, error bars represent SEM. (B) Mice immunized with MS2 L9 (15aa) and MS2 L9 (27aa) received a third immunization, given seven weeks after the prime. Data compares antibody responses after either two or three immunizations. (C) Anti-CSP antibody responses in mice immunized twice with unadjuvanted MS2 L9 (15aa) VLPs compared to mice that received two doses MS2 L9 (15aa) plus Cquim-MA adjuvant and mice that received a Qβ L9 prime (plus Cquim-MA) followed by an MS2 L9 (15aa) boost (also with Cquim-MA). Sera were obtained one week following the second immunization.

FIG. 3. Longevity of antibody responses. (A) Groups of Balb/c mice (n=5) were immunized intramuscularly with 5 μg of VLPs at weeks 0, 3, and 7 and were measured following each immunization and at 38-47 weeks post-prime. Anti-CSP IgG titers were calculated by end-point dilution ELISA. Geometric means plus SEM are shown at each timepoint. (B) Anti-CSP IgG titers were calculated by end-point dilution ELISA for mice that received vaccine plus Cquim-MA adjuvant received two immunizations, one at week 0 and a second at week 3.

FIG. 4. Both Qβ L9 and MS2 L9 (15aa) VLPs plus Cquim-MA elicit strong antibody responses and inhibit malaria infection. (A) Experimental timeline. C57BL/6 mice were immunized three times at weeks 0, 3, and 6, followed by a challenge with five Pb-PfCSP-Luc-infected mosquitoes at week eight. Liver luminescence was measured 42 hours after the mosquito challenge, and blood smears were taken starting on day 3 post-infection. (B) Anti-CSP antibody levels in serum obtained following the third vaccination, prior to challenge. Horizontal lines represent the mean anti-CSP antibody concentration for each group. Groups are compared by 2-tailed unpaired t test. (C) Parasite liver burden measured via luminescence. Horizontal lines indicate geometric mean luminescence for each group. Statistical comparisons were performed using a two-tailed Mann Whitney test. (D) Extent of blood-stage infection, as measured by percent of blood parasite-free mice post-challenge. Log-rank test was used to statistically compare protected groups to naïve mice.

FIG. 5. A combination vaccine targeting the L9 epitope provides the strongest inhibition of malaria infection. (A) Anti-CSP antibody levels in serum obtained following the third vaccination. Horizontal lines represent the mean anti-CSP antibody concentration for each group. No significant differences were observed between groups (p>0.05, unpaired t-test). (B) Parasite liver burden measured via luminescence. Horizontal lines indicate geometric mean luminescence for each group. Statistical comparisons were performed using a two-tailed Mann Whitney test. (C) Extent of blood-stage infection, as measured by percent of blood parasite-free mice post-challenge. Log-rank test was used to statistically compare protected groups to naïve mice.

### DETAILED DESCRIPTION OF ILLUSTRATIVE EMBODIMENTS

This disclosure describes antigenic malaria circumsporozoite protein (CSP) peptides useful in making a vaccine against malaria, methods of preparing a vaccine that includes an antigenic CSP peptide, and methods of treatment that include administering an antigenic CSP peptide vaccine to a subject.

Malaria is a significant global public health concern. A disproportionate share of malarial disease and deaths occurs in Africa and is caused by infection with the Plasmodium falciparum parasite. P. falciparum (Pf) infection is initiated when the Anopheles mosquito injects sporozoites into the blood stream of a human host. Sporozoites are transported quickly to the liver where they transiently multiply within hepatocytes, producing merozoites, which then enter the blood stream where they invade red blood cells (RBCs), replicate further, and cause the symptoms and pathology of malaria.

Several different malaria vaccine strategies have been proposed, including vaccines that target transmission, the erythrocytic stage in which symptoms occur, and the pre-erythrocytic stage. A pre-erythrocytic vaccine that effectively blocks malaria infection of hepatocytes could potentially provide sterilizing immunity against malaria. However, development of such a vaccine has been complicated by a number of factors, including (1) natural immunity to the pre-erythrocytic stage is weak and ineffective, (2) surface antigens expressed on sporozoites are antigenically variable, and (3) high titers of circulating antibody are likely required to effectively inhibit infection.

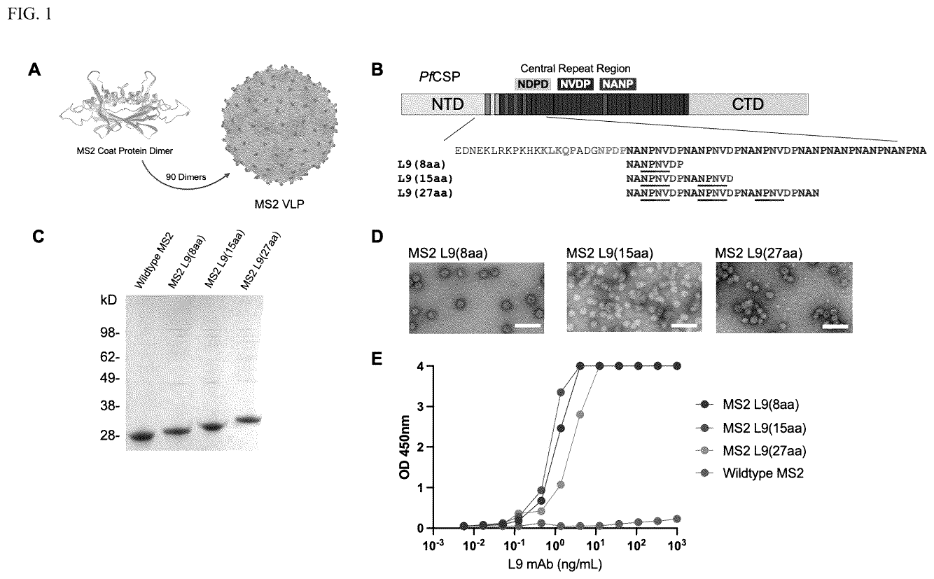

Most attempts to develop pre-erythrocytic vaccines have targeted the malaria circumsporozoite protein (CSP). CSP is the most abundant protein on the surface of the malaria sporozoite and is an attractive vaccine target because anti-CSP antibodies reduce the likelihood and/or extent that the malaria parasite reaches the liver and establishes infection. Malarial vaccines targeting the central repeat (CR) region of the CSP (FIG. 1B) only moderately reduce human infection. For example, CSP is the target of RTS,S/AS01_{E} (also referred to as RTS,S), the most advanced malaria vaccine. However, the RTS,S vaccine confers only moderate (30-50%) reduction of clinical infection and immunity rapidly declines. Thus, there remains a need for a malaria vaccine with higher potency and provides more durable immune responses than the RTS,S vaccine.

Human monoclonal antibodies (mAbs) from human volunteers immunized with an experimental irradiated whole sporozoite vaccine (PfSPZ) that target amino acid residues in the CSP but outside of the central repeat region have shown the ability to reduce malaria infection in animal models, which points to new sites of vulnerability in CSP that may be exploited using epitope-targeted vaccines.

Most previous CSP-targeted vaccines have used full-length or near full-length antigen. RTS,S, for example, contains a large domain of the CSP central-repeat region and most of its C-terminal domain. While a subset of antibodies elicited by RTS,S reduce infection, using large domains of an antigen can be problematic as critical epitopes may be hidden or immunologically subdominant.

U.S. Pat. No. 12,161,710 discloses a bacteriophage Qβ-based virus-like particle (VLP) vaccine that multivalently displays a peptide recognized by the CIS43 mAb, which is located at the junction between the N-terminal region of CSP and the CR, and can elicit extremely durable and high-titer anti-CSP antibody responses, and reduce parasite liver burden in a mouse malaria challenge model, but did not prevent blood-stage parasitemia. U.S. Pat. No. 12,121,574 discloses a Qβ-based virus-like particle (VLP) vaccine that multivalently displays a 15-amino-acid peptide (SEQ ID NO:3) recognized by the L9 mAb and can elicit extremely durable and high-titer anti-CSP antibody responses, reduce parasite liver burden in a mouse malaria challenge model, and reduce blood-stage parasitemia.

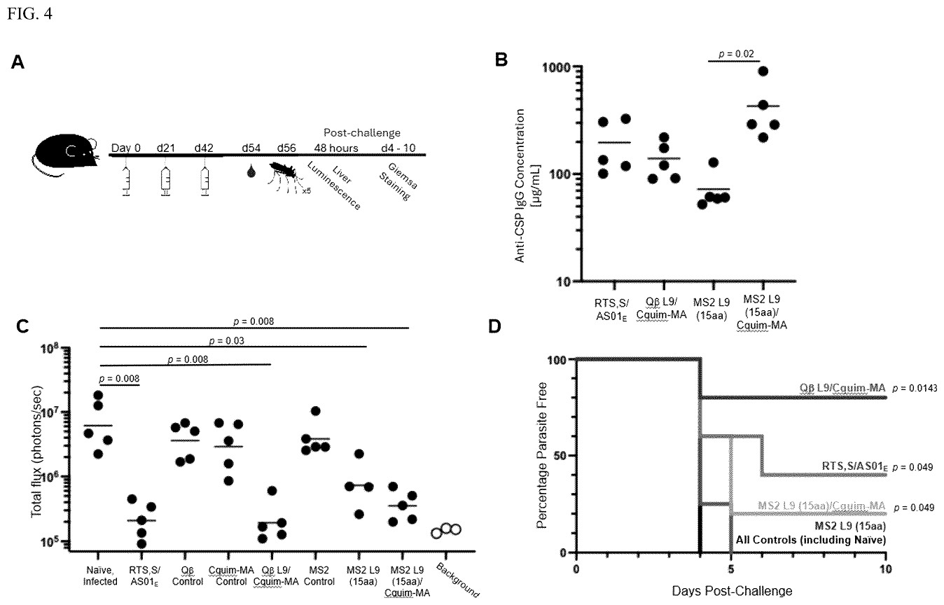

This disclosure describes the development and characterization of a recombinant bacteriophage VLP-based vaccine targeting the amino acid residues recognized by the L9 mAb, a previously described anti-CSP antibody (Wang et al. Immunity 53, 733-744 e738 (2020)). The amino acids recognized by the L9 mAb (SEQ ID NO:3) overlap with the amino acids recognized by the CIS43 mAb (amino acids 21-35 of SEQ ID NO:1), but is centered on the minor repeat sequences at the N-terminus of the central repeat region (FIG. 1B). Despite their similarity in sequence, the L9-based vaccine described herein was able to reduce parasite liver load (FIG. 4C) and reduce blood stage parasitemia (FIG. 4D) in a malaria challenge experiment.

### VLP Display

Many viral structural proteins have an intrinsic ability to self-assemble into virus-like particles (VLPs), which structurally resemble the virus from which they were derived but, because they lack viral genomes, they are absolutely noninfectious. VLPs not only can serve as stand-alone vaccines, but because their particulate nature and multivalent structure provoke strong immune responses, they can be used as platforms to enhance the immunogenicity of heterologous antigenic targets. For example, when short immunogenic peptides are displayed in a highly repetitive, multivalent fashion on VLPs, peptide-specific B cells are strongly activated, leading to high-titer, long-lasting antibody responses. VLPs derived from diverse virus types can serve as effective platforms for antigen display. The immunogens described herein are based on VLPs derived from a family of related single-stranded RNA bacteriophages, including MS2, PP7, AP205, and Qβ. These VLPs can be produced by expressing a single viral structural protein, called coat, from a plasmid in a bacterium. Peptides may be displayed on a VLP by engineering recombinant coat proteins that contain the target epitope or by using bioconjugation techniques that employ cross linker molecules. In one or more embodiments, a peptide may be displayed on a VLP by inserting a target peptide into a surface-exposed structure on coat protein such that the resulting VLP displays the target at high valency (e.g., 90 peptides per VLP) on its surface. In one or more embodiments, a peptide may be displayed on a VLP by conjugating the peptide to the VLP through a succinimidyl-6-[β-maleimidopropionamido]hexanoate (SMPH) cross-linker molecule. This technique results in VLPs that display target peptides at high valency, usually 180-360 peptides per VLP. Regardless of the strategy employed, the VLP platform confers strong immunogenicity to displayed immunogenic peptides.

### Adjuvant Platforms

While alum remains the dominant adjuvant used in human vaccines worldwide, it has shown poor utility in malaria vaccines, with oil emulsions and more complex adjuvant combinations such as AS01 delivering more favorable results in human studies. However, oil emulsion adjuvants are associated with high reactogenicity and the ability of the RTS,S/AS01 vaccine to reduce infection attenuates rapidly. A major malaria vaccine challenge is to find a suitable adjuvant platform that overcomes these obstacles. This adjuvant platform needs to be able to drive strong and long-lasting humoral and cellular immune responses while being non-reactogenic and safe for use in young children.

Cquim-MA (ViroVax, LLC) is a dual TLR7/8 agonist-based adjuvant that is designed to traffic to draining secondary lymphoid tissue, where it interacts with antigen. Thus, Cquim-MA can exert adjuvant activity while limiting the extent to which it exists systemically.

ADVAX (Vaxine Pty, Ltd., Adelaide, Australia) was developed as a polysaccharide particle adjuvant platform based on the plant sugar, delta inulin (Petrovsky et al., 2015, Vaccine 33:5920-5926) to further enhance its adjuvant potency, ADVAX can be co-formulated with a toll-like receptor (TLR) agonist such as a CpG oligonucleotide (e.g., CpG55.2) that activates TLR9 and/or an imidazoquinoline amine that activates TLR7 and/or TLR8. This results in highly effective vaccine formulations that can induce immunity sufficient to inhibit infection after just one immunization, generate longer lasting and more broadly cross-neutralizing antibodies, and production of durable memory CD4 and CD8 T cell responses (Petrovsky et al., 2015, Vaccine 33:5920-5926). These adjuvant formulations can be designed to shape the immune response in any desired direction, e.g., Th1, Th2, or Th17, that correlates best with immunity. Further, these adjuvants can be formulated with one or more additional ingredients such as, for example, aluminum hydroxide.

### Construction and Assembly of MS2 VLPs Displaying L9 Peptides

The coat protein of bacteriophage MS2 can be engineered so that it can tolerate insertion of heterologous peptide sequences in a surface exposed loop on its surface. This display site, which is referred to as the AB loop, has a beta-hairpin structure that protrudes from the surface of the virus particle (FIG. 1A). Given that the core NPNV (SEQ ID NO: 11) L9 epitope adopts a type 1 beta-turn, displaying this epitope in a more relevant structural context could elicit more potent antibody responses. Nucleotide sequences encoding peptides representing a minimal 8-amino acid L9 epitope (SEQ ID NO:2) centered around the core NPNV (SEQ ID NO: 11) sequence; the 15-amino acid epitope (SEQ ID NO:3), which contains two NPNV (SEQ ID NO:11) sequences; and an extended 27 amino acid epitope (SEQ ID NO:4) were inserted into the downstream AB-loop of the MS2 coat protein single-chain dimer expression vector. Expression plasmids were then used to express recombinant MS2 L9 VLPs in E. coli. Because each standard MS2 VLP has 90 copies of the single-chain dimer, each recombinant MS2 VLP can display 90 copies of the L9 peptide per VLP. Successful insertion of the L9 peptides was confirmed by SDS-PAGE analysis. Coat proteins from recombinant MS2 L9 VLPs displayed a higher molecular weight compared to wild-type MS2 coat protein (FIG. 1C). To confirm that the insertion of different L9 peptides into the MS2 coat protein was compatible with VLP assembly, transmission electron microscopy (TEM) was used to visualize the structures of the three different MS2 L9 VLPs (FIG. 1D). These results showed that all three of the MS2 L9 constructs assembled into particles with diameters of approximately 30 nm. The morphology of MS2 VLPs displaying the longer 15-amino acid and 27-amino acid L9 sequences was somewhat less regular than VLPs displaying the shorter 8-amino acid peptide.

The antigenicity of MS2 L9 VLPs was assessed by measuring the binding of L9 mAb to the MS2 L9 VLPs by enzyme-linked immunosorbent assay (ELISA). All three MS2 L9 VLPs were strongly recognized by the L9 mAb (FIG. 1E), whereas wild-type MS2 VLPs were not. These findings confirm that displaying L9 epitopes in a constrained loop structure on MS2 VLPs does not affect L9 mAb binding.

### MS2 L9 VLPs Elicit Strong Anti-CSP Antibody Responses

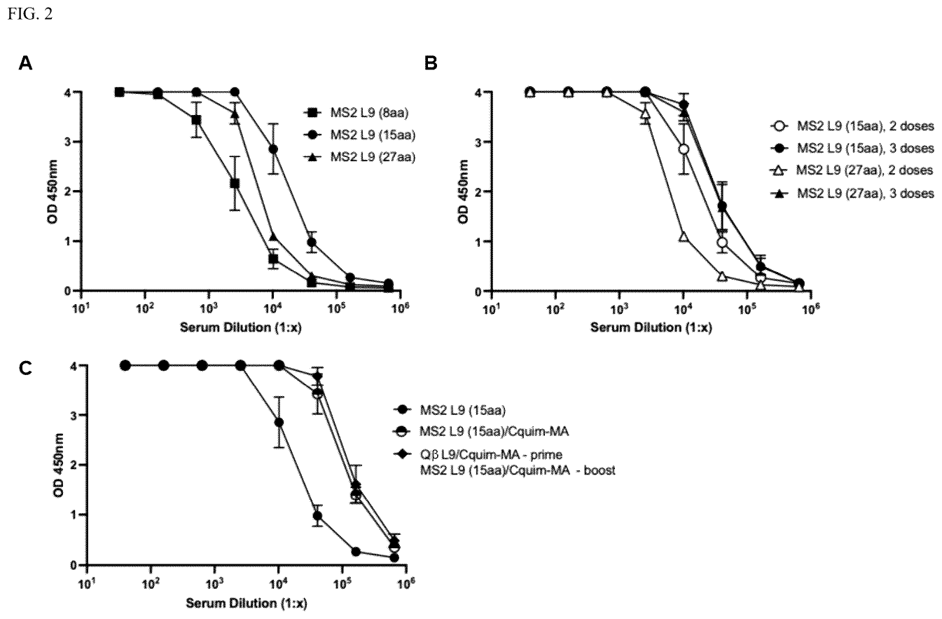

The immunogenicity of the three MS2 L9 VLPs constructs was compared in mice. Mice were initially immunized twice, at week 0 and week 3, and sera were collected one week after the second immunization to measure antibody levels against full-length recombinant CSP by ELISA. All three MS2 L9 VLPs elicited strong anti-CSP IgG responses, with end-point dilution titers ranging from 4×10⁴ to 6.4×10⁵ (FIG. 2A). MS2 L9 (15aa) VLPs elicited the highest titer anti-CSP antibody responses. Based on their improved immunogenicity compared to the VLPs displaying the L9 (8aa) epitope, MS2 L9 VLPs displaying the longer (15aa and 27aa) epitopes were selected for further investigation. In each of these groups, mice received a third immunization to determine whether an additional boost would further enhance the immune response. This third immunization resulted in a substantial increase in anti-CSP antibody levels in mice that received the MS2 L9 (27aa) VLPs, and a modest increase in anti-CSP antibody levels in mice immunized with MS2 L9 (15aa) VLPs (FIG. 2B). After three immunizations, the antibody responses induced by the MS2 L9 (27aa) and MS2 L9 (15aa) VLPs were nearly identical (FIG. 2B).

To evaluate if adjuvants could enhance antibody responses to MS2-based vaccines, the anti-CSP antibody responses generated by MS2 L9 (15aa) VLPs formulated with Cquim-MA, a dual TLR7/8 agonist adjuvant, were measured. Two immunizations with MS2 L9 (15aa) VLPs plus Cquim-MA induced markedly stronger anti-CSP antibody responses compared with unadjuvanted MS2 L9 (15aa) VLPs (FIG. 2C).

Heterologous prime-boost regimens can be an effective method for focusing antibody responses against specific epitope targets and minimizing antibody responses against the vaccine platform. To determine if this approach could also be used to boost anti-CSP antibody responses, mice were primed with Qβ L9 VLPs mixed with Cquim-MA and then boosted with MS2 L9 (15aa) VLPs, also formulated with Cquim-MA. This heterologous prime-boost approach resulted in similar anti-CSP antibody responses compared to those induced by MS2 L9 (15aa) VLPs with Cquim-MA (FIG. 2C). Taken together, these data demonstrate that MS2 L9 VLPs can elicit exceptionally strong anti-CSP antibody responses with or without heterologous boosting, especially in combination with Cquim-MA.

### MS2 L9 VLPs Elicit Durable Antibody Responses

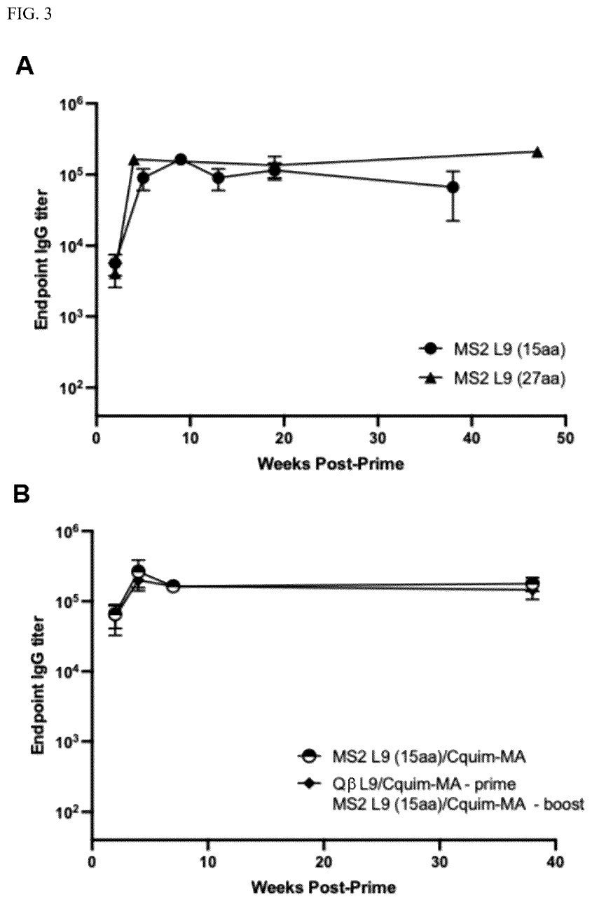

One advantage of VLP-based vaccines is that they reliably elicit durable antibody responses, likely through the efficient induction of long-lived plasma cells. To assess the duration of the anti-CSP antibody response elicited by MS2 L9 VLPs, mice received three immunizations of MS2 L9 (15aa) or MS2 L9 (27aa) and antibody responses were measured for nearly a year following the prime. Both vaccines elicited long-lived antibodies, with little to no reduction in geometric mean titers over 40-50 weeks following the initial prime (FIG. 3A). Similarly, the durability of antibody responses in mice that received two doses of Cquim-MA adjuvanted vaccines was compared (FIG. 3B). Mice received either a homologous prime-boost with MS2 L9 (15aa) or a heterologous prime-boost with Qβ L9 followed by MS2 L9 (15aa). Both vaccine regimens elicited similar peak antibody titers (following the second immunization). Antibody levels slightly declined from the peak titer (immediately following the second immunization) to the final timepoint (38 weeks following the prime). Use of Cquim-MA with MS2 L9 (15aa) slightly increased antibody titers compared to unadjuvanted vaccine at the late (38 week) timepoint, but this difference was not statistically significant.

Immunization with MS2 L9 (15Aa) VLPs Plus Cquim-MA Adjuvant Inhibits Infection Following Malaria Challenge in Mice

The extent to which MS2 L9 VLPs could inhibit infection following malaria challenge was evaluated in a mouse model. Vaccine efficacy was assessed in a model in which vaccinated mice were challenged with mosquitoes carrying transgenic Plasmodium berghei (Pb) sporozoites expressing full-length PfCSP and luciferase (Pb-PfCSP-Luc). In this model, parasite liver loads can be quantified by measuring luciferase signal in the liver. In addition, the ability of vaccination to mediate sterilizing immunity can be determined by monitoring the development of blood-stage infection (parasitemia). In the initial challenge experiment (FIG. 4A), mice were immunized with MS2 L9 (15aa) VLPs and monitored for malaria infection. Mice received three immunizations with MS2 L9 (15aa) VLPs, with or without Cquim-MA. To benchmark vaccine efficacy, groups of mice were also immunized three times with Qβ L9 VLPs plus Cquim-MA or with the WHO-approved vaccine RTS,S/AS01_{E} (using a 5 μg dose). As negative controls, groups of mice were immunized with wild-type MS2 VLPs (not displaying the L9 epitope), wild-type Qβ VLPs, or Cquim-MA adjuvant alone, or were unimmunized (naïve). Following immunization, anti-CSP antibody concentrations from individual mice were quantified. As shown in FIG. 4B, RTS,S/AS01_{E}, Qβ L9/Cquim-MA, MS2 L9 (15aa), and MS2 L9 (15aa)/Cquim-MA induced strong anti-CSP antibody responses. MS2 L9 (15aa)/Cquim-MA elicited the highest anti-CSP antibody concentrations, with mean levels approximately six-fold higher than unadjuvanted MS2 L9 (15aa), approximately three-fold higher than Qβ L9/Cquim-MA, and approximately 2.2-fold higher than RTS,S/AS01_{E}. None of the control groups elicited anti-CSP antibodies (not shown).

Mice were then infected by exposure to five Pb-PfCSP-Luc infected mosquitoes. As an initial determination of vaccine efficacy, liver-stage parasite burden was quantified by measuring liver luciferase activity using intravital imaging 42 hours post-infection (FIG. 4C). Immunization with RTS,S/AS01_{E} dramatically reduced parasite liver burden, by approximately 97% relative to naïve controls. Liver-stage parasite burden in mice immunized with Qβ L9/Cquim-MA was similar to the liver-stage parasite burden in mice immunized with RTS,S/AS01_{E}. Mice immunized with MS2 L9 (15aa) displayed an 88% reduction in parasite liver load while mice immunized with MS2 L9 (15aa)/Cquim-MA displayed a 94% reduction in parasite liver load. None of the negative control groups showed a significant reduction in infection (FIG. 4C).

To evaluate the possibility of sterilizing immunity, daily blood smears were taken from the challenged mice starting at day 3 post-infection. Blood was assessed for parasitemia using Giemsa staining (FIG. 4D). While all control groups developed blood-stage parasitemia by day 4, 40% of the RTS,S/AS01_{E}-vaccinated group (two out of five mice) were parasite free while 80% of the mice immunized Qβ L9/Cquim-MA were completely parasite free, which is an improvement compared to previous studies (U.S. Pat. No. 12,121,574) using Qβ L9 adjuvanted with ADVAX-3 (Vaxine Pty, Ltd., Adelaide, Australia), suggesting that Cquim-MA may be a more effective adjuvant in combination with this vaccine. Although MS2 L9 (15aa)/Cquim-MA elicited the highest anti-CSP antibody concentrations, only 20% mice immunized with this vaccine remained free from blood-stage parasitemia.

### A Combination L9 VLP Vaccine Reduces Liver Parasite Burden and Enhances Sterilizing Immunity in Plasmodium-Challenged Mice

A second challenge experiment was performed with the following goals: (1) to replicate the prior results showing that vaccination with Qβ L9/Cquim-MA provides stronger protection than RTS,S/AS01_{E}, (2) to evaluate the efficacy of MS2 L9 VLPs displaying the longer 27aa L9 peptide, and (3) to assess protection of a combination of the MS2 L9 (15aa) and Qβ L9 vaccines. Mice were immunized with RTS,S/AS01, Qβ L9/Cquim-MA, MS2 L9 (27aa)/Cquim-MA, or a mixture of Qβ L9 and MS2 L9 (15aa)/Cquim-MA. Negative controls included a group of mice that received wildtype Qβ VLPs and a group of unimmunized (naïve) mice.

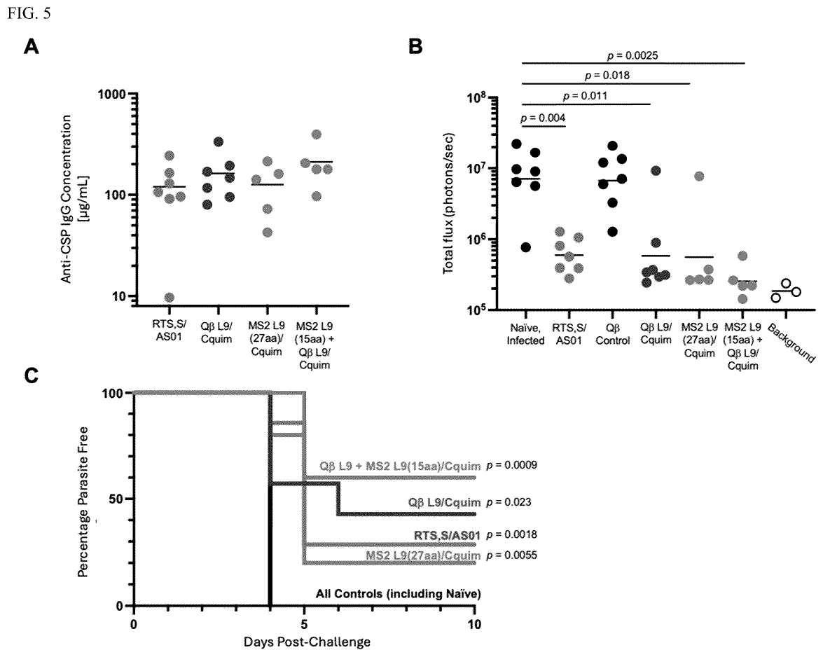

The Qβ-based L9 vaccine and the MS2-based L9 vaccines, adjuvanted with Cquim-MA, elicited strong anti-CSP antibody responses, with mean levels>100 μg/mL, similar to the antibody levels induced by RTS,S/AS01_{E} (FIG. 5A). Immunization with Qβ L9/Cquim-MA or MS2 L9 (27aa)/Cquim-MA resulted in similar reductions in parasite liver loads as RTS,S/AS01 (~91%), but mice that received the combination vaccine plus Cquim-MA showed the greatest reduction in parasite burden (~96%) (FIG. 5B). Similar to the initial challenge experiment (FIG. 4), vaccination with Qβ L9/Cquim-MA resulted in sterilizing immunity in a higher percentage of mice than were observed in the group immunized with RTS,S/AS01_{E} (FIG. 5C). The percentage of mice that were parasite free was lower in this experiment (43%) compared to the previous challenge study (80%). This may be explained by the higher mean parasite liver burden across all groups in the second challenge experiment (for example, it was 20% higher in the naïve group). Immunization with the combination vaccine (Qβ L9 VLPs plus MS2 L9 VLPs with Cquim-MA) provided the strongest sterilizing immunity, with a higher percentage of mice exhibiting sterilizing immunity than in mice immunized with either RTS,S/AS01_{E} or Qβ L9/Cquim-MA. Taken together, these results indicate that a combination vaccine presenting the L9 epitope in multiple conformations, as an unstructured peptide on Qβ VLPs and in a structured β-hairpin on MS2 VLPs, was highly effective at eliciting sterilizing immunity against Plasmodium infection.

Thus, this disclosure describes a VLP-based immunogen that specifically targets CSP. These particles are highly immunogenic in a mouse model and elicit a long-lived antibody response. To increase the immunogenicity of L9 VLPs, the L9 VLPs can be combined with an adjuvant. Suitable adjuvants include, but are not limited to, carbomeriquim (Cquim-MA), delta inulin polysaccharide-based ADVAX adjuvants, or any other standard adjuvant. In particular, combining L9 VLPs with Cquim-MA yields higher anti-CSP antibody titers than unadjuvanted L9 VLPs.

While described herein in the context of exemplary embodiments in which the VLP platform used to present the antigenic CSP peptide is an MS2 VLP, the compositions and methods described herein can involve the use of any suitable VLP platform. Thus, VLPs that present an antigenic CSP peptide can be derived from any one of a family of related single-stranded RNA bacteriophages including, but not limited to, MS2, PP7, AP205, or Qβ.

The VLP-based immunogen includes an antigenic CSP peptide (also referred to herein as a “a CSP-targeting peptide”) such as, for example, the amino acids of SEQ ID NO:2, 9-14 contiguous amino acids of SEQ ID NO:3, SEQ ID NO:3, or a structurally similar peptide. Further, the immunogen can include a VLP that displays more than one population of antigenic CSP peptides—e.g., a first population of antigenic CSP peptides that includes the amino acids of SEQ ID NO:2, 9-14 contiguous amino acids of SEQ ID NO:3, SEQ ID NO:3 and a second population of antigenic CSP peptides. The second population of CSP peptides can include, but are not limited to, SEQ ID NO:2, 9-14 contiguous amino acids of SEQ ID NO:3, SEQ ID NO:3, or any other immunogenic CSP peptide described in U.S. Pat. No. 12,161,710 or U.S. Pat. No. 12,121,574, or a peptide structurally similar to any of the foregoing. Thus, the immunogen can be designed to display one, two, three, four, five, six, or more antigenic CSP peptides.

In another aspect, an immunogenic composition may include more than one population of VLPs. For example, an immunogenic composition can include a first population of VLPs displaying the antigenic CSP peptide including the amino acids SEQ ID NO:2 (or a structurally similar peptide) and a second population of VLPs displaying a second antigenic CSP peptide. The second antigenic CSP peptide can include, but is not limited to, SEQ ID NO:2, 9-14 contiguous amino acids of SEQ ID NO:3, SEQ ID NO:3, or any other immunogenic CSP peptide described in U.S. Pat. No. 12,161,710 or U.S. Pat. No. 12,121,574, or a peptide structurally similar to any of the foregoing. As another example, an immunogenic composition may include a first population of VLPs displaying one or more antigenic CSP peptides and a second population of VLPs displaying one or more antigenic CSP peptides, independent of the number and identity of the antigenic peptides displayed by the first population of VLPs.

As used herein, a peptide is “structurally similar” to a reference polypeptide if the amino acid sequence of the peptide possesses a specified amount of identity compared to the reference peptide. Structural similarity of two peptides can be determined by aligning the residues of the two peptides (for example, a candidate polypeptide and, e.g., SEQ ID NO:2, SEQ ID NO:3, or a fragment of SEQ ID NO:3) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate peptide is the peptide being compared to the reference peptide (e.g., SEQ ID NO:2, SEQ ID NO:3, or a fragment of SEQ ID NO:3). A candidate peptide can be isolated, for example, from an animal, or can be produced using recombinant techniques, or chemically or enzymatically synthesized.

A pair-wise comparison analysis of amino acid sequences can be carried out using the BESTFIT algorithm in the GCG package (version 10.2, Madison WI). Alternatively, peptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

An antigenic CSP peptide can include amino acids in addition to the amino acids of SEQ ID NO:2, 9-14 contiguous amino acids of SEQ ID NO:3, SEQ ID NO:3, or any other immunogenic CSP peptide described in U.S. Pat. No. 12,161,710 or U.S. Pat. No. 12,121,574, or a peptide structurally similar to any of the foregoing, so long as the additional amino acids do not eliminate immunogenicity toward CSP. For example, an antigenic CSP peptide may have a linker region containing the amino acids GGGC (SEQ ID NO: 5), the amino acids CGGG (SEQ ID NO:6), or any other suitable linker sequence.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also includes the presence of conservative substitutions. A conservative substitution for an amino acid in an immunogenic peptide as described herein may be selected from other members of the class to which the amino acid belongs. For example, it is well-known in the art of protein biochemistry that an amino acid belonging to a grouping of amino acids having a particular size or characteristic (such as charge, hydrophobicity, and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gin for Asn to maintain a free —NH₂. Likewise, biologically active analogs of a polypeptide containing deletions or additions of one or more contiguous or noncontiguous amino acids that do not eliminate a functional activity of the peptide are also contemplated.

In one or more embodiments, a CSP-targeting peptide as described herein can include a peptide with at least 50%, at least 62.5%, at least 75%, or at least 82.5% sequence similarity to the amino acids of SEQ ID NO:2. That is, a CSP-targeting polypeptide can include a total of no more than four, no more than three, no more than two, or no more than one amino acid deletions and non-conservative amino acid substitutions compared to SEQ ID NO:2.

In one or more embodiments, a CSP-targeting peptide as described herein can include a peptide with at least 50%, at least 62.5%, at least 75%, or at least 82.5% sequence identity to SEQ ID NO:2. That is, a CSP-targeting polypeptide can include a total of no more than four, no more than three, no more than two, or no more than one amino acid deletions and amino acid substitutions compared to SEQ ID NO:2.

In one or more embodiments, a CSP-targeting peptide as described herein can include a peptide with at least 60%, at least 66%, at least 73%, at least 80%, at least 86%, or at least 93% sequence similarity to amino acids SEQ ID NO:3. That is, a CSP-targeting polypeptide can include a total of no more than six, no more than five, no more than four, no more than three, no more than two, or no more than one amino acid deletions and non-conservative amino acid substitutions compared to SEQ ID NO:3.

In one or more embodiments, a CSP-targeting peptide as described herein can include a peptide with at least 60%, at least 66%, at least 73%, at least 80%, at least 86%, or at least 93% sequence identity to SEQ ID NO:3. That is, a CSP-targeting polypeptide can include a total of no more than six, no more than five, no more than four, no more than three, no more than two, or no more than one amino acid deletions and amino acid substitutions compared to SEQ ID NO:3.

In one or more embodiments, a CSP-targeting peptide as described herein can be a fragment of SEQ ID NO:3 that includes, at a minimum, the amino acids of SEQ ID NO:2. Thus, a CSP-targeting peptide can include at least nine contiguous amino acids of SEQ ID NO:3, at least 10 contiguous amino acids of SEQ ID NO:3, at least 11 contiguous amino acids of SEQ ID NO:3, at least 12 contiguous amino acids of SEQ ID NO:3, at least 13 contiguous amino acids of SEQ ID NO:3, or at least 14 contiguous amino acids of SEQ ID NO:3.

In one or more embodiments, a CSP-targeting peptide as described herein can be designed to provide additional sequences, such as, for example, the addition of added C-terminal or N-terminal amino acids that can, for example, facilitate purification by trapping on columns or use of antibodies. Such tags include, for example, histidine-rich tags that allow purification of polypeptides on nickel columns. Such gene modification techniques and suitable additional sequences are well known in the molecular biology arts.

The virus-like particle (VLP) can include any particle that includes viral protein assembled to structurally resemble the virus from which they are derived, but lack enough of the viral genome so that they are non-replicative and, therefore, noninfectious. A VLP may, therefore, include at least some of the viral genome, but the viral genome is genetically modified so that the viral genes responsible for infectivity and replication are inactivated. Exemplary VLPs include, but are not limited to, VLPs of Qβ, MS2, PP7, AP205, or other bacteriophage coat proteins, the capsid and core proteins of Hepatitis B virus, measles virus, Sindbis virus, rotavirus, foot-and-mouth-disease virus, Norwalk virus, the retroviral GAG protein, the retrotransposon Ty protein pl, the surface protein of Hepatitis B virus, human papilloma virus, human polyoma virus, RNA phages, Ty, frphage, GA-phage, AP 205-phage and, in particular, Qβ-phage, Cowpea chlorotic mottle virus, cowpea mosaic virus, human papilloma viruses (HPV), bovine papilloma viruses, porcine parvovirus, parvoviruses such as B19, porcine (PPV) and canine (CPV) parvovirues, caliciviruses (e.g. Norwalk virus, rabbit hemorrhagic disease virus [RHDV]), animal hepadnavirus core Antigen VLPs, filamentous/rod-shaped plant viruses, including but not limited to Tobacco Mosaic Virus (TMV), Potato Virus X (PVX), Papaya Mosaic Virus (PapMV), Alfalfa Mosaic Virus (AIMV), and Johnson Grass Mosaic Virus (JGMV), insect viruses such as flock house virus (FHV) and tetraviruses, polyomaviruses such as Murine Polyomavirus (MPyV), Murine Pneumotropic Virus (MPtV), BK virus (BKV), and JC virus (JCV).

The antigenic CSP peptides may be coupled to immunogenic carriers via chemical conjugation or by expression of genetically engineered fusion partners. The coupling does not necessarily need to be direct, but can occur through linker sequences. More generally, when an antigenic peptide is either fused, conjugated, or otherwise attached to an immunogenic carrier, spacer sequence, or linker sequence, the immunogenic carrier, spacer sequence, or linker sequence is typically added at one or both ends of the antigenic peptide. Such linker sequences generally include sequences recognized by the proteasome, proteases of the endosomes, or other vesicular compartment of the cell.

In one embodiment, the antigenic CSP peptide may be displayed as fusion protein with a subunit of the immunogenic carrier. Fusion of the peptide can be effected by inserting the CSP antigenic peptide amino acid sequence into the immunogenic carrier primary sequence, or by fusion to either the N-terminus or C-terminus of the immunogenic carrier.

When the immunogenic carrier is a VLP, the chimeric antigenic peptide-VLP subunit can be capable of self-assembly into a VLP. VLP displaying epitopes fused to their subunits are also herein referred to as chimeric VLPs. For example, European Application No. EP90310264A (European Patent No. EP0421635 B1) describes the use of chimeric hepadnavirus core antigen particles to present foreign peptide sequences in a virus-like particle.

Flanking amino acid residues may be added to either end of the sequence of the antigenic peptide to be fused to either end of the sequence of the subunit of a VLP, or for internal insertion of such peptide sequence into the sequence of the subunit of a VLP. Glycine and serine residues are particularly favored amino acids to be used in the flanking sequences added to the peptide to be fused. Glycine residues confer additional flexibility, which may diminish the potentially destabilizing effect of fusing a foreign sequence into the sequence of a VLP subunit.

In one or more embodiments, the immunogenic carrier is a VLP of a RNA phage such as, for example, MS2 or Qβ. The major coat proteins of RNA phages spontaneously assemble into VLPs upon expression in bacteria such as, for example, E. coli. Foreign peptides, such as an antigenic CSP peptide, can be inserted into a site within the coat protein that tolerates a peptide insertion without disrupting the ability of the coat protein to properly fold and assemble into a VLP. In one or more embodiments, the insertion site can be within the AB-loop on the surface of coat protein. Peptides inserted within the AB-loop are prominently displayed on the surface of the VLP.

Coat protein of MS2 normally folds as a dimer, ninety of which assemble into the icosahedral MS2 VLP. The wild-type form of MS2 coat protein is highly intolerant of peptide insertions in the AB-loop, with the vast majority (usually >98%) leading to folding failures. However, an engineered form of MS2 coat protein renders it more tolerant of AB-loop insertions (Caldeira et al., 2010. Vaccine 28(27):4384-4393; Peabody et al., 2008, J Mol Biol 380, 252-263). The engineered form of MS2 exploits the proximity of the N-terminus and the C-terminus of the two identical polypeptide chains in the dimer. By duplicating the coat protein coding sequence and then fusing the two copies into a single reading frame, the engineered form of MS2 acts as a so-called single-chain dimer. This form of the protein is more stable thermodynamically and its folding is more tolerant of peptides inserted into the AB-loop of the downstream copy of the single-chain dimer. Inserting heterologous peptides, such as the L9 sequences described herein, into the AB-loop of the single-chain dimer of the MS2 coat protein results in the formation of VLPs that display one peptide per dimer, or ninety peptides per VLP.

Further VLPs suitable for fusion of antigens or antigenic determinants are described in, for example, International Patent Application No. PCT/IB2002/004132 (International Publication No. WO 03/024481 A2) and include bacteriophage fr, RNA phage PP7, capsid protein of papillomavirus, retrotransposon Ty, yeast and also Retrovirus-like-particles, HIV2 Gag, Cowpea Mosaic Virus, parvovirus VP2 VLP, HBsAg (U.S. Pat. No. 4,722,840). Examples of chimeric VLPs suitable for use as the immunogenic carrier include those described in Kozlovska et al., 1996, Intervirology 39:9-15. Further examples of VLPs suitable for use as the immunogenic carrier include, but are not limited to, HPV-1, HPV-6, HPV-11, HPV-16, HPV-18, HPV-33, HPV-45, CRPV, COPV, HIV GAG, Tobacco Mosaic Virus, Virus-like particles of SV-40, Polyomavirus, Adenovirus, Herpes Simplex Virus, Rotavirus, and Norwalk virus.

In one or more embodiments, a vaccine construct containing a CSP peptide is synthesized by inserting a CSP sequence into the AB-loop of the single-chain dimer version of the MS2 coat protein. In this methodology, a plasmid containing this recombinant MS2 coat protein is expressed in E. coli and then recombinant MS2 VLPs displaying the CSP epitope are isolated and purified.

For any recombinantly expressed antigenic CSP peptide described herein (whether or not coupled to an immunogenic carrier), this disclosure describes the nucleic acid that encodes the peptide or protein, as is an expression vector comprising the nucleic acid, and a host cell containing the expression vector (autonomously or chromosomally inserted). This disclosure further describes a method of recombinantly producing the peptide or protein by expressing it in a host cell with or without further isolating the immunogen.

Thus, this disclosure describes an isolated nucleic acid sequence that encodes any embodiment of an antigenic CSP peptide described herein. In one or more embodiments, the isolated polynucleotide encodes an immunogenic portion of SEQ ID NO:1 such as, for example, the amino acid sequence of SEQ ID NO:2, the amino acid sequence of SEQ ID NO:3, or the amino acid sequence of a fragment of SEQ ID NO:3 (e.g., a fragment containing 9-14 contiguous amino acids of SEQ ID NO:3). Given the amino acid sequence of any immunogenic CSP peptide, a person of ordinary skill in the art can determine the full scope of polynucleotides that encode that amino acid sequence using conventional, routine methods.

As used herein, the term “nucleic acid,” “polynucleotide,” or “oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugates, and oligonucleotides. A polynucleotide may be single-stranded, double-stranded, linear, or covalently circularly closed molecule. A polynucleotide can be isolated. The term “isolated polynucleotide” means that the polynucleotide (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A polynucleotide might be introduced—i.e., transfected—into cells. When RNA is used to transfect cells, the RNA may be modified by stabilizing modifications, capping, or polyadenylation.

As used herein “amplified DNA” or “PCR product” refers to an amplified fragment of DNA of defined size. Various techniques are available and well known in the art to detect PCR products. PCR product detection methods include, but are not restricted to, gel electrophoresis using agarose or polyacrylamide gel and adding ethidium bromide staining (a DNA intercalant), labeled probes (radioactive or non-radioactive labels, southern blotting), labeled deoxyribonucleotides (for the direct incorporation of radioactive or non-radioactive labels) or silver staining for the direct visualization of the amplified PCR products; restriction endonuclease digestion, which relies on agarose gel electrophoresis, polyacrylamide gel electrophoresis, or high-performance liquid chromatography (HPLC); dot blots, using the hybridization of the amplified DNA on specific labeled probes (radioactive or non-radioactive labels); high-pressure liquid chromatography using ultraviolet detection; electro-chemiluminescence coupled with voltage-initiated chemical reaction/photon detection; and direct sequencing using radioactive or fluorescently labeled deoxyribonucleotides for the determination of the precise order of nucleotides with a DNA fragment of interest, oligo ligation assay (OLA), PCR, qPCR, DNA sequencing, fluorescence, gel electrophoresis, magnetic beads, allele specific primer extension (ASPE) and/or direct hybridization.

Generally, a polynucleotide can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; and 9,012,208. Examples of nucleic acid analysis include, but are not limited to, sequencing and DNA-protein interaction. Sequencing may be by any method known in the art. DNA sequencing techniques include classic dideoxy sequencing reactions (Sanger method) using labeled terminators or primers and gel separation in slab or capillary, and next generation sequencing methods such as sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes.

This disclosure also describes a host cell including any of the isolated nucleic acid sequences and/or proteins described herein. Thus, this disclosure encompasses translation of a polynucleotide (e.g., an mRNA) by a host cell to produce an immunogenic CSP peptide and/or a VLP that displays an immunogenic CSP peptide.

The polynucleotide constructs of the present invention may be introduced into a host cell to be altered, thus allowing expression of the CSP peptide and/or CSP VLP within the cell, thereby generating a genetically engineered cell. A variety of methods are known in the art and suitable for introducing a polynucleotide into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as LIPOFECTAMINE (Thermo Fisher Scientific, Inc., Waltham, MA), HILYMAX (Dojindo Molecular Technologies, Inc., Rockville, MD), FUGENE (Promega Corp., Madison, WI), JETPEI (Polyplus Transfection, Illkirch, France), EFFECTENE (Qiagen, Hilden, Germany) and DreamFect (OZ Biosciences, Inc USA, San Diego, CA).

The polynucleotide constructs described herein may be introduced into a host cell to be altered, thus allowing expression within the cell of the protein encoded by the nucleic acid. A variety of host cells are known in the art and suitable for protein expression. Examples of typical cells used for transfection and protein expression include, but are not limited to, a bacterial cell, a eukaryotic cell, a yeast cell, an insect cell, or a plant cell such as, for example, E. coli, Bacillus, Streptomyces, Pichia pastoris, Salmonella typhimurium, Drosophila S2, Spodoptera SJ9, CHO, COS (e.g., COS-7), 3T3-F442A, HeLa, HUVEC, HUAEC, NIH 3T3, Jurkat, 293, 293H, or 293F.

In one or more embodiments, the antigenic CSP peptide can be chemically coupled to the immunogenic carrier using techniques well known in the art. Conjugation can occur to allow free movement of peptides via single point conjugation (e.g., either N-terminal or C-terminal point) or as a locked down structure where both ends of peptides are conjugated to either an immunogenic carrier protein or to a scaffold structure such as a VLP. Conjugation occurs via conjugation chemistry known to those skilled in the art such as via cysteine residues, lysine residues, or another carboxy moiety. Thus, for example, for direct covalent coupling, it is possible to use a carbodiimide, glutaraldehyde, or N-[y-maleimidobutyryloxy]succinimide ester, using common commercially available hetero-bifunctional linkers such as 1-cyano-4-dimethylaminopyridinium tetrafluoroborate (CDAP) or succinimidyl 3-(2-pyridyldithio)propionate (SPDP).

Examples of conjugation of peptides, particularly cyclized peptides, to a protein carrier via acylhydrazine peptide derivatives are described in, for example, International Patent Application No. PCT/EP2003/004551 (International Publication No. WO 2003/092714 A1). After the coupling reaction, the immunogen can easily be isolated and purified using, for example, a dialysis method, a high performance liquid chromatography method, a gel filtration method, a fractionation method, etc. Peptides terminating with a cysteine residue (preferably with a linker outside the cyclized region) may be conveniently conjugated to a carrier protein via maleimide chemistry.

When the immunogenic carrier is a VLP, several antigenic peptides, either having an identical amino acid sequence or a different amino acid sequence, may be coupled to a single VLP particle, leading preferably to a repetitive and ordered structure presenting several antigenic determinants in an oriented manner as described in International Patent Applications PCT/IB1999/001925 (International Publication No. WO 00/032227), PCT/IB2002/004132 (International Publication No. WO 2003/024481), PCT/IB2002/000166 (International Publication No. WO 02/056905), and PCT/EP2003/007572 (International Publication No. WO 2004/007538). Thus, the antigenic peptide displayed by one VLP subunit in a VLP may be the same or different than the antigenic peptide displayed by a second VLP subunit in the same VLP. In other embodiments, one or several antigen molecules can be attached to one VLP subunit. A specific feature of the VLP of the coat protein of RNA phages and, particularly, of the MS2 coat protein VLP is thus the possibility to couple several antigens per subunit. This allows for the generation of a dense antigen array.

Another feature of VLPs derived from RNA phage is their high expression yield in bacteria that allows production of large quantities of material at affordable cost. Moreover, the use of the VLPs as carriers allows the formation of robust antigen arrays and conjugates, respectively, with variable antigen density. The use of VLPs of RNA phages, and particularly the use of the VLP of RNA phage MS2 coat protein, allows a very high antigen density to be achieved.

### Compositions and Methods of Treatment

The CSP-targeting VLP may be used to treat a subject having, or at risk of having, a condition characterized, at least in part, by cells that express CSP. Such conditions include, but are not limited to, malaria.

As used herein, “treat” or variations thereof refer to reducing, limiting progression, ameliorating, or resolving, to any extent, the symptoms or signs related to a condition. A “sign” or “clinical sign” refers to an objective physical finding relating to a particular condition capable of being found by one other than the patient. A “symptom” refers to any subjective evidence of disease or of a patient's condition.

A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject. Typically, the CSP-targeted VLP will be used prophylactically to reduce the likelihood that the malaria parasite reaches the liver.

Treatment that is prophylactic—e.g., initiated before a subject manifests a symptom or clinical sign of the condition such as, for example, while a tumor remains subclinical—is referred to herein as treatment of a subject that is “at risk” of having the condition. As used herein, the term “at risk” refers to a subject that may or may not actually possess the described risk. Thus, for example, a subject “at risk” of developing a condition is a subject possessing one or more risk factors associated with the condition such as, for example, genetic predisposition, ancestry, age, sex, geographical location, lifestyle, or medical history. Thus, the CSP-targeted VLP may be administered before a subject manifests a symptom or clinical sign of malaria. In one or more embodiments, the CSP-targeted VLP may be administered before a subject travels to a geographical location where malaria may be prevalent.

Accordingly, a composition can be administered before, during, or after the subject first exhibits a symptom or clinical sign of the condition (e.g., malaria). Treatment initiated before the subject first exhibits a symptom or clinical sign associated with the condition may result in decreasing the likelihood that the subject experiences clinical evidence of the condition compared to a subject to which the composition is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

Thus, the method includes administering an effective amount of the composition to a subject having, or at risk of having, a condition characterized, at least in part, by cells that express CSP. In this aspect, an “effective amount” is an amount effective to reduce, limit progression, ameliorate, or resolve, to any extent, a symptom or clinical sign related to the condition.

Thus, the CSP-targeting MS2 VLP described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the CSP-targeting MS2 VLP without causing any undesirable biological effects or interacting in a deleterious manner with any of the other components of the pharmaceutical composition in which it is contained.

The CSP-targeting MS2 VLP may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, a CSP-targeting MS2 VLP may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant (whether Cquim-MA or other adjuvant), a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the CSP-targeting MS2 VLP into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of CSP-targeting MS2 VLP administered can vary depending on various factors including, but not limited to, the cancer being treated, the weight, physical condition, and/or age of the subject, and/or the route of administration. Thus, the absolute weight of CSP-targeting MS2 VLP included in a given unit dosage form can vary widely, and can depend upon factors such as the species, age, weight, and physical condition of the subject, and/or the method of administration. Accordingly, it is not practical to set forth generally the amount that constitutes an amount of CSP-targeting MS2 VLP effective for all possible applications. Those of ordinary skill in the art, however, can readily determine the appropriate amount with due consideration of such factors.

In one or more embodiments, the method can include administering sufficient CSP-targeting MS2 VLP to provide a dose of, for example, from about 50 ng/kg to about 1 mg/kg to the subject, although in one or more embodiments the methods may be performed by administering CSP-targeting MS2 VLP in a dose outside this range.

In one or more embodiments, the method includes administering sufficient CSP-targeting MS2 VLP to provide a minimum dose of at least 50 ng/kg such as, for example, at least 100 ng/kg, at least 200 ng/kg, at least 300 ng/kg, at least 400 ng/kg, at least 500 ng/kg, at least 600 ng/kg, at least 700 ng/kg, at least 800 ng/kg, at least 900 ng/kg, at least 1 μg/kg, at least 2 μg/kg, at least 5 μg/kg, at least 10 μg/kg, at least 20 μg/kg, at least 50 μg/kg, at least 100 μg/kg, at least 200 μg/kg, or at least 500 μg/kg.

In one or more embodiments, the method includes administering sufficient CSP-targeting MS2 VLP to provide a maximum dose of no more than 1 mg/kg, no more than 500 μg/kg, no more than 250 μg/kg, no more than 200 μg/kg, no more than 150 μg/kg, no more than 100 μg/kg, no more than 50 μg/kg, no more than 25 μg/kg, no more than 10 μg/kg, no more than 5 μg/kg, no more than 2 μg/kg, no more than 1 μg/kg, no more than 800 ng/kg, no more than 600 ng/kg, no more than 500 ng/kg, no more than 400 ng/kg, no more than 300 ng/kg, no more than 250 ng/kg, no more than 150 ng/kg, no more than 100 ng/kg, no more than 50 ng/kg, or no more than 25 ng/kg.

In one or more embodiments, the method includes administering sufficient CSP-targeting MS2 VLP to provide a dose that falls within a range having as endpoints any minimum dose listed above and any maximum dose listed above that is greater than the minimum does. For example, in one or more embodiments, the method can include administering sufficient CSP-targeting MS2 VLP to provide a dose of from 200 ng/kg to about 10 μg/kg to the subject, for example, a dose of from about 700 ng/kg to about 5 μg/kg.

In one or more embodiments, CSP-targeting MS2 VLP may be administered, for example, from a single dose to multiple doses per week, although in one or more embodiments the method can be performed by administering CSP-targeting Qβ VLP at a frequency outside this range. When multiple doses are used within a certain period, the amount of each dose may be the same or different. For example, a dose of 1 mg per day may be administered as a single dose of 1 mg, two 0.5 mg doses, or as a first dose of 0.75 mg followed by a second dose of 0.25 mg. Also, when multiple doses are used within a certain period, the interval between doses may be the same or be different.

In certain embodiments, CSP-targeting MS2 VLP may be administered at minimum frequency of at least once per year such as, for example, at least once every six months, at least once every four months, at least once every three months, at least once every two months, at least once per month, or at least once every two weeks.

In certain embodiments, CSP-targeting MS2 VLP may be administered at maximum frequency of no more than once per week such as, for example, no more than once every two weeks, no more than once per month, no more than once every two months, no more than once every three months, no more than once every six months, or once per year.

In one or more embodiments, CSP-targeting MS2 VLP may be administered at a frequency defined by a range having as endpoints any minimum frequency listed above and any maximum frequency listed above that is more frequent than the selected minimum frequency.

The duration of administration of an antigenic CSP peptide described herein, e.g., the period of time over which an antigenic CSP peptide is administered, can vary, depending on any of a variety of factors, e.g., patient response, etc. For example, an antigenic CSP peptide can be administered over a period of time ranging from about one day to about one week, from about two weeks to about four weeks, from about one month to about two months, from about two months to about four months, from about four months to about six months, from about six months to about eight months, from about eight months to about one year, from about one year to about two years, or from about two years to about four years, or more. In one or more embodiments, the CSP-targeting MS2 VLP may be administered as a once off treatment. In other embodiments, the CSP-targeting MS2 VLP may be administered for the life of the subject. In certain embodiments, the CSP-targeting MS2 VLP may be administered monthly (or every four weeks) until effective.

In one or more embodiments, the CSP-targeting MS2 VLP may be administered at an initial frequency for an initial period and then administered at a lower frequency thereafter. For example, a dosing regimen may include administering three doses of the CSP-targeting MS2 VLP at a frequency of once per month (i.e., an initial dose followed by a second dose one month after the initial dose) followed by an additional dose six months after the initial dose.

When a CSP-targeting MS2 VLP composition is used for prophylactic treatment, it may be generally administered for priming and/or boosting doses. Boosting doses, when administered, are adequately spaced (e.g., yearly) to boost the level of circulating antibody that has fallen below a desired level. Boosting doses may include a CSP-targeting peptide either with or in the absence of the original immunogenic carrier. A booster composition may include an alternative immunogenic carrier or may be in the absence of any carrier. Moreover, a booster composition may be formulated either with or without adjuvant.

In some cases, the method can further include administering to the subject an additional therapeutic agent effective for treating the condition (e.g., malaria). For example, therapy involving the CSP-targeting MS2 VLP may be combined with conventional therapies for malaria.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

As used herein, “have,” “has,” “having,” “include,” “includes,” “including,” “comprise,” “comprises,” “comprising” or the like are used in their open-ended inclusive sense, and generally mean “include, but not limited to,” “includes, but not limited to,” or “including, but not limited to.” Further, wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, that which follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

As used herein, the word “exemplary” means to serve as an illustrative example and should not be construed as preferred or advantageous over other embodiments.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

In several places throughout the above description, guidance is provided through lists of examples, which examples can be used in various combinations. In each instance, the recited list serves only as a representative group and should not be interpreted as an exclusive list.

For any method disclosed herein that includes discrete steps, the steps may be performed in any feasible order. And, as appropriate, any combination of two or more steps may be performed simultaneously.

### EXAMPLES

The present invention is illustrated by the following examples. It is to be understood that the particular examples, materials, amounts, and procedures are to be interpreted broadly in accordance with the scope and spirit of the invention as set forth herein.

### Production and Characterization of Recombinant MS2 VLPs

The MS2 VLP expression plasmid pDSP62, which encodes the single-chain dimer version of the MS2 bacteriophage coat protein, was generated as previously described (Chackerian et al., 2011, J Mol Biol 409, 225-237; Peabody et al., 2008, J Mol Biol 380, 252-263). Sequences encoding the L9 epitopes (SEQ ID NO:2, SEQ ID NO:3, SEQ ID NO:4) were generated by PCR and cloned into pDSP62 so that the L9 epitope was inserted into the surface-exposed AB-loop of the downstream copy of the MS2 bacteriophage coat protein. Cloned constructs were sequenced to confirm the presence of the sequence encoding the L9 epitope.

MS2 VLPs displaying L9 epitopes were produced as previously described (Mogus et al., 2020, Vaccines (Basel) 8). Briefly, expression plasmids were used to transform the C41 E. coli expression strain by electroporation. Transformed C41 cells were grown at 37° C. using Luria Bertani broth containing 50 μg/mL kanamycin until the cells reached an OD600 of 0.6. Coat protein expression was induced using 0.4 mM isopropyl-β-D-1-thiogalactopyranoside and grown at 37° C. overnight. Cell pellets were collected, re-suspended in lysis buffer [50 mM Tris-HCL, 100 mM NaCl, 10 mM ethylenediaminetetraacetic acid, pH 8.5, 0.45% deoxycholate] and incubated on ice for 30 minutes. Cells were lysed by 3-5 cycles of sonication at 20 Hz and then cell lysates were clarified by centrifugation (15,000×g, 20 minutes, 4° C.). Soluble MS2 L9 VLPs were purified by precipitation using 70% saturated (NH₄)₂SO₄, followed by size exclusion chromatography (SEC) using a Sepharose CL-4B column. The column was pre-equilibrated with a purification buffer (40 mM Tris-HCl, 400 mM NaCl, 8.2 mM MgSO₄, pH 7.4). MS2 L9 VLPs were concentrated from SEC purified fractions by precipitation using 70% saturated (NH₄)₂SO₄ followed by dialysis against PBS (pH 7.4). Endotoxin was removed from preparations by three rounds of Triton X-114 (Sigma-Aldrich) phase separation, using a previously described protocol (Aida et al., 1990, J Immunol Methody 132, 191-195). The final concentration and purity of VLPs was determined via SDS-PAGE using a 10% NuPAGE gel (Invitrogen).

Visualization of the VLPs with transmission electron microscope (TEM) was performed as previously described (Tumban et al., 2011, PLoS One 6). VLPs were adsorbed on carbon-coated glow-discharged copper grids for two minutes and then were negatively stained with 2% uranyl acetate for two minutes. VLPs were visualized at a magnification of 100,000×.

The display of L9 epitopes on the surface of recombinant VLPs was confirmed by ELISA. Briefly, 250 ng of MS2 L9 VLPs were coated onto wells of an ELISA plate (IMMUNULON 2, Thermo Fisher Scientific, Inc., Waltham, MA) and probed with serial dilutions of the L9 monoclonal antibody (L9 mAb), provided by Robert Seder at the NIH Vaccine Research Center. L9 mAb binding was detected using a 1:4000 dilution of horseradish peroxidase (HRP)-conjugated goat anti-human IgG (Jackson ImmunoResearch, West Grove, PA), followed by the addition of 3,3′,5,5′-tetramethylbenzidine (TMB) substrate (Thermo Fisher Scientific, Inc., Waltham, MA). The reaction was stopped by the addition of 1% HCl, and optical density was measured at 450 nm (OD450) using a plate reader (ACCUSKAN, Fisher Scientific International, Inc., Hampton, NH).

### Production of QR L9 VLPs

Qβ L9 VLPs were generated by chemically conjugating a synthetic peptide representing the CSP L9 epitope to preformed Qβ VLPs, as previously described previously (Peabody et al., 2008, J Mol Biol 380, 252-263). Briefly, a peptide representing the L9 epitope of CSP, modified to contain the C-terminal linker sequence gly-gly-gly-cys (SEQ ID NO:5) (NANPNVDPNANPNVDGGGC, SEQ ID NO:7), was synthesized by GenScript Biotech Corp. (Nanjing, China). L9 peptides were conjugated to exposed lysine residues on the surface of Qβ VLPs by using the heterobifunctional crosslinker succinimidyl 6-[(beta-maleimidopropionamido) hexanoate](SMPH; Thermo Fisher Scientific, Inc., Waltham, MA). SMPH was incubated with Qβ VLPs at a molar ratio of 10:1 (SMPH:Qβ coat protein) for two hours at room temperature. Excess SMPH was removed using a centrifugal unit with a 100 kDa cutoff (AMICON Ultra-4, MilliporeSigma, Burlington, MA). L9 peptide was incubated with Qβ VLPs at a molar ratio of 10:1 (peptide: Qβ coat protein) and allowed to react overnight at 4° C.

### Mouse Immunization and Challenge Studies

To assess vaccine immunogenicity, groups of 4-5-week-old female Balb/c mice (typically n=5 per group; Jackson Laboratory) were immunized intramuscularly with 5 μg of MS2 L9 VLPs or control VLPs. When adjuvant was used, vaccines were mixed with 5 μg of carbomeriquim (Cquim-MA), a dual TLR7/8 agonist (ViroVax LLC, Lawrence, KS). Mice were boosted with the same dose of vaccine and adjuvant three and seven weeks after the initial prime. Sera were collected one week after each immunization and, in some cases, monthly thereafter.

For challenge studies, groups of 7-8-week-old C57Bl/6 mice were used. Mice were immunized with 5 μg of VLPs with or without 2 μg Cquim-MA adjuvant. As negative controls, mice were immunized with wild-type (unmodified) VLPs, Cquim-MA adjuvant alone, or were unimmunized. As benchmark controls, mice received 5 μg of RTS,S/AS01. Vaccine was administered on days 0, 28, and 56. Sera were collected following the final boost (at day 82) and then mice were challenged two days later.

Mice were challenged using Anopheles stephensi mosquitoes infected with transgenic P. berghei sporozoites engineered to express luciferase and full-length PfCSP in place of P. berghei CSP, denoted as Pb-PfCSP-luc (Flores-Garcia et al., 2019, Malar J 18, 426). Infected mosquitoes were generated by allowing the insects to blood-feed on Pb-PfCSP-Luc-infected mice. Prior to exposure, mice were anesthetized with 2% tribromoethanol. Each mouse was then exposed to five infected mosquitoes for a 10-minute blood meal. After feeding, the number of mosquitoes with visible blood meals was recorded. Liver luminescence was measured 42 hours post-challenge. Mice were anesthetized, and 100 μL of D-luciferin (30 mg/mL) was administered via intraperitoneal injection. Luminescence from the liver was subsequently captured using an imaging system (IVIS Spectrum, PerkinElmer, Shelton, CT). Starting four days post-challenge, blood smears were collected daily and stained with Giemsa to monitor parasitemia.

### Quantification of Antibody Responses

Anti-CSP antibody responses were quantified by ELISA as reported previously in Jelinkova et al., 2021, NPJ Vaccines 6, 13 and Jelinkova et al., 2022, NPJ Vaccines 7, 34. To measure serum antibody levels against full-length CSP, an ELISA was performed using recombinant CSP, expressed in Pseudomonas fluorescens (Noe et al., PLoS One. 2014 Sep. 23; 9(9):e107764). ELISA plates (IMMIULON 2, Thermo Fisher Scientific) were coated with 250 ng of recombinant CSP in 50 μL of PBS per well overnight at 4° C. or for two hours at room temperature (RT). Wells were blocked with 100 μL PBS-0.5% nonfat dry milk for one hour at RT or overnight at 4° C. Sera isolated from immunized mice were serially diluted in PBS-0.5% milk and applied to wells for two hours at RT or overnight at 4° C. Reactivity was measured by adding HRP-labeled goat anti-mouse IgG, diluted 1:4000 in PBS-0.5% milk (Jackson ImmunoResearch, West Grove, PA) for one hour at RT, and detected by adding TMB substrate. Reactions were stopped using 1% HCl and optical density was measured at 450 nm (OD450) using a plate reader (ACCUSKAN, Fisher Scientific International, Inc., Hampton, NH). For some challenge experiments, CSP-specific antibody concentrations in mouse sera were quantified by linear regression analysis using a standard curve generated using known concentrations of the anti-CSP mouse monoclonal antibody 2A10, as previously described (Jelinkova et al., 2021, NPJ Vaccines 6, 13 and Jelinkova et al., 2022, NPJ Vaccines 7, 34).

### Statistical Analysis

All statistical analyses were conducted using PRISM v.9 (GraphPad Software, Inc., San Diego, CA). Unpaired two-tailed t-tests were used for comparing antibody levels and Mann-Whitney tests were used for the mosquito challenge experiments. For percent inhibition calculations, liver luminescence values of individual vaccinated mice were divided by the mean of mice in negative control groups. Background luminescence levels were subtracted from all values.

The complete disclosure of all patents, patent applications, and publications, and electronically available material (including, for instance, nucleotide sequence submissions in, e.g., GenBank and RefSeq, and amino acid sequence submissions in, e.g., SwissProt, P PRF, PDB, and translations from annotated coding regions in GenBank and RefSeq) cited herein are incorporated by reference in their entirety. In the event that any inconsistency exists between the disclosure of the present application and the disclosure(s) ofany document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Sequence Listing Free Text

SEQ ID NO: 1
EDNEKLRKPK HKKLKQPADG NPDPNANPNV DPNANPNVDP NANPNVDPNA NPNANPNANP
NANPNA
SEQ ID NO: 2 (8 amino acid L9 sequence)
NANPNVDP
SEQ ID NO: 3 (15 amino acid L9 sequence)
NANPNVDPNA NPNVD
SEQ ID NO: 4 (27 amino acid L9 sequence)
NANPNVDPNA NPNVDPNANP NVDPNAN
SEQ ID NO: 5
GGGC
SEQ ID NO: 6
CGGG
SEQ ID NO: 7
NANPNVDPNA NPNVDGGGC
SEQ ID NO: 8
NDPD
SEQ ID NO: 9
NVDP
SEQ ID NO: 10
NANP
SEQ ID NO: 11
NPNV

What is claimed is:

1. An immunogen comprising:
an immunogenic carrier comprising an MS2 bacteriophage virus-like particle (VLP); and
an antigenic malaria circumsporozoite protein (CSP) peptide comprising NANPNVDP (SEQ ID NO:2) linked to the immunogenic carrier.

2. An immunogen comprising:
an immunogenic carrier comprising a bacteriophage VLP; and
an antigenic malaria CSP peptide linked to the immunogenic carrier;
wherein the CSP peptide comprises:
amino acids 1 to 8 of SEQ ID NO:3,
amino acids 1 to 9 of SEQ ID NO:3,
amino acids 1 to 10 of SEQ ID NO:3,
amino acids 1 to 11 of SEQ ID NO:3,
amino acids 1 to 12 of SEQ ID NO:3,
amino acids 1 to 13 of SEQ ID NO:3, or
amino acids 1 to 14 of SEQ ID NO:3.

3. The immunogen of claim 2, wherein the bacteriophage VLP comprises an MS2 bacteriophage VLP or a Qβ bacteriophage VLP.

4. The immunogen of any one of claim 1, further comprising a second antigenic malaria CSP peptide.

5. The immunogen of claim 4, wherein the first CSP peptide and the second antigenic CSP peptide are displayed on a single VLP.

6. The immunogen of claim 4, wherein the second antigenic CSP peptide comprises amino acids 21-35 of SEQ ID NO:1.

7. A composition comprising the immunogen of claim 1.

8. The composition of claim 7, comprising:
a first population of VLPs displaying SEQ ID NO:2; and
a second population of VLPs displaying a second antigenic CSP peptide.

9. The composition of claim 7, further comprising an adjuvant.

10. The composition of claim 9, wherein the adjuvant comprises an agonist of Toll-like receptor (TLR)7, an agonist of TLR8, or an agonist of both TLR7 and TLR8.

11. A method of treating malaria in an individual, the method comprising administering a therapeutically effective amount of a composition to the individual, the composition comprising the immunogen of claim 1.

12. The method of claim 11, wherein the method further comprises administering to the individual at least one additional therapeutic agent for treating malaria.

13. The method of claim 11, wherein the composition comprises:
a first population of immunogens comprising the immunogen of claim 1; and
a second population of immunogens comprising:
a second population of immunogenic carriers; and
the second CSP peptide linked to the second population of immunogenic carriers.

14. The method of claim 11, wherein the composition is administered to the individual:
before the individual manifests a symptom or clinical sign of malaria, or
before the individual travels to a geographical location where malaria may be prevalent.

15. A polynucleotide encoding the immunogen of claim 1.

16. A vaccine comprising the composition of claim 7.

17. A method of treating malaria in an individual, the method comprising administering to the individual a therapeutically effective amount of the vaccine of claim 16.

18. The method of claim 17, wherein the method further comprises administering to the individual at least one additional therapeutic agent for treating malaria.

19. The method of claim 17, wherein the vaccine is administered to the individual:
before the individual manifests a symptom or clinical sign of malaria, or
before the individual travels to a geographical location where malaria may be prevalent.

20. A method of treating Plasmodium falciparum blood stage parasitemia in an individual, the method comprising administering to the individual a therapeutically effective amount of the composition of claim 7.

21. The method of claim 20, wherein the method further comprises administering to the individual at least one additional therapeutic agent for treating malaria.

22. The method of claim 20, wherein the composition is administered to the individual before the individual manifests a symptom or clinical sign of Plasmodium falciparum blood stage parasitemia.


SEQUENCE LISTING (11 sequences)

SEQ ID NO: 1 - protein, 66 aa, synthetic construct
EDNEKLRKPKHKKLKQPADGNPDPNANPNVDPNANPNVDPNANPNVDPNANPNANPNANPNANPNA

SEQ ID NO: 2 - protein, 8 aa, synthetic construct
NANPNVDP

SEQ ID NO: 3 - protein, 15 aa, synthetic construct
NANPNVDPNANPNVD

SEQ ID NO: 4 - protein, 27 aa, synthetic construct
NANPNVDPNANPNVDPNANPNVDPNAN

SEQ ID NO: 5 - protein, 4 aa, synthetic construct
GGGC

SEQ ID NO: 6 - protein, 4 aa, synthetic construct
CGGG

SEQ ID NO: 7 - protein, 19 aa, synthetic construct
NANPNVDPNANPNVDGGGC

SEQ ID NO: 8 - protein, 4 aa, synthetic construct
NDPD

SEQ ID NO: 9 - protein, 4 aa, synthetic construct
NVDP

SEQ ID NO: 10 - protein, 4 aa, synthetic construct
NANP

SEQ ID NO: 11 - protein, 4 aa, synthetic construct
NPNV